(19) United States
(12) Patent Application Publication
(10) Pub. No.: US 2014/0066465 A1
(43) Pub. Date: Mar. 6, 2014

(54) CANCER TREATMENT USING TYROSINE KINASE AND NF-kB INHIBITORS

(71) Applicant: The Cleveland Clinic Foundation, Cleveland, OH (US)

(72) Inventors: George R. Stark, Shaker Heights, OH (US); Kam Tai Dermawan, Shaker Hts., OH (US)

(73) Assignee: The Cleveland Clinic Foundation, Cleveland, OH (US)

(21) Appl. No.: 14/017,938

(22) Filed: Sep. 4, 2013

Related U.S. Application Data
(60) Provisional application No. 61/696,444, filed on Sep. 4, 2012.

(51) Int. Cl. A61K 31/473; A61K 31/517

(52) U.S. Cl. CPC A61K 31/473; A61K 31/517
USPC 514/266.4

(57) ABSTRACT

A method of treating cancer by administering to a subject in need thereof a therapeutically effective amount of a tyrosine kinase inhibitor and an NF-κB inhibitor is described. The method is useful for treating subjects having cancer that has developed resistance to tyrosine kinase inhibitors.

### CROSS-REFERENCE TO RELATED APPLICATIONS

This Application claims priority to U.S. Provisional Patent Application Ser. No. 61/696,444, filed Sep. 4, 2012, which is incorporated herein by reference.

### STATEMENT ON FEDERALLY FUNDED RESEARCH

The present invention was supported, at least in part, by government support by the Nation Institutes of Health under Grant No. CA 095851. The Government has certain rights in this invention.

### BACKGROUND

Lung cancer remains the leading cause of cancer death in the United States, with approximately 222,520 new cases diagnosed and 157,300 deaths in 2010. Non-small cell lung cancer makes up approximately 80% of all lung cancers and is comprised of multiple histologic subtypes. The major NSCLC subtypes are squamous carcinoma, adenocarcinoma, and large cell carcinoma. While patients with early stage disease may be cured by surgery or surgery with adjuvant chemotherapy, cure in patients with unresectable disease is rarely seen. Patients with recurrent/metastatic disease may achieve improved survival and palliation of symptoms with platinum-based chemotherapy (survival rates of about 35% at 1 year.

First-line treatment for metastatic or recurrent NSCLC usually involves platinum-based chemotherapy doublets, and about 25-35% of patients treated with one of these chemotherapy combinations achieve a response that lasts 4-5 months. In patients with EGFR mutation (EGFRmut(+) patients), who comprise 10-30% of all NSCLC, as detected by the FDA-approved cobas EGFR mutation test, erlotinib is the first-line treatment until disease progression, which typically occurs at 9-13 months due to second-site EGFR T790M mutation or Met amplification, or as yet unknown mechanisms. Patients receiving second-line treatment for advanced NSCLC have options of either EGFR inhibitor (erlotinib) or further chemotherapy (docetaxel or pemetrexed). Second-line treatment with docetaxel improved efficacy compared with best supportive care or other single-agent chemotherapies. Erlotinib delays disease progression and increases survival after first-line chemotherapy in patients with advanced NSCLC as second-line therapy or as maintenance therapy. A recent study compared second line erlotinib with standard chemotherapy regimens (docetaxel or pemetrexed). Results from this study showed no significant differences in efficacy between patients treated with erlotinib or standard chemotherapy and better adverse effect profile for erlotinib.

Erlotinib is a tyrosine kinase inhibitor (TKI) that is currently approved as a first-line therapy in NSCLC with EGFR mutation, monotherapy in patients with locally advanced or metastatic non-small cell lung cancer after failure of at least one prior chemotherapy regimen (Shepherd et al. N Engl J Med., 353(2):123-132 (2005)), or in combination with gemcitabine for first line treatment in patients with locally advanced, unresectable or metastatic pancreatic cancer. Moore et al. J Clin Oncol., 25(15):1960-1966 (2007). However, the majority of patients with advanced NSCLC do not have activating, TKI-sensitizing EGFR mutations and they only derive a modest benefit from TKIs; even the initially sensitive EGFR-mut(+) patients typically will eventually develop resistance to treatment with tyrosine kinase inhibitors through further mutation of EGFR or through activation of downstream survival pathways via MET amplification. Kobayashi et al., N Engl J Med., 352(8):786-92 (2005). In addition, FAS and NF-κB signaling have been shown to modulate dependence of lung cancers on mutant EGFR. Bivona et al., Nature, 471(7339):523-526 (2011).

Quinacrine was widely used during World War II as antimalarial agent. It is no longer used for this purpose due to development of better drugs with more desirable properties. Quinacrine Hydrochloride (ATABRINE®) was manufactured in the USA by Winthrop-Breon (Sanofi-Winthrop) Laboratories in the form of 100 mg tablets, 1950-1990's. In 1992, Sanofi-Winthrop discontinued Atabrine's (quinacrine) production in the United States due to commercial reasons. Over the last four decades quinacrine was used in the treatment of giardiasis, tapeworm infestations and connective tissue diseases (lupus erythematosus, rheumatoid arthritis). The drug has also been used for chemical pleurodesis for recurrent pleural effusion in cancer patients. Quinacrine is currently undergoing prospective evaluation for management of Creutzfeldt-Jakob disease (CJD).

A chemical library has been screened for compounds that will activate wild type p53. Using a kidney cancer cell line with inactive but wild type p53 and a p53-responsive reporter as a readout system, a diverse chemical library was screened. Compounds that were capable of restoring p53 transactivation in RCC cells were isolated. Restoration of p53 function resulted in selective killing of tumor cells in a p53-dependent manner and differentially from conventional chemotherapeutic drugs. Structural analysis of a subset of isolated chemicals revealed 9-aminoacridine (9AA) as the chemical group critical for p53 activation. Gurova et al., Proc Natl Acad Sci USA., 102(48):17448-17453 (2005).

The most promising characteristics of 9AA included: (a) very strong activation of p53 in almost all tumor cells tested (with wild type p53) (b) p53 dependent cytoxicity for tumor cells, in contrast to normal cells, and (c) a new mechanism of p53 activation. 9AA activates p53 by inhibition of NF-κB. NF-κB is a transcription factor regulating expression of pro-inflammatory and anti-apoptotic proteins. Its activity was shown to be responsible for resistance to many types of cellular stresses, including DNA damage, reactive oxygen species, hypoxia, and death-ligands induced apoptosis. It is frequently constitutively active in tumor cells, in contrast to normal cells, in which it is activated in response to certain pro-inflammatory stimuli. NF-κB is considered a target of therapeutic inhibition in cancer. An inverse correlation between NF-κB and p53 activity has been noted in several systems and is usually described as “a swing”—high activity of either of factors leads to suppression of another. Webster G A, Perkins N D., Mol Cell Biol., 19(5):3485-3495 (1999). 9AA converts NF-κB into a transrepressive form through inhibition of phosphorylation of one of the main NF-κB subunits, p65. Such a mechanism of activity of 9AA makes it a potent inhibitor of not only stimulated activity of NF-κB, which is characteristic of IKK2 inhibitors, but also against basal activity of NF-κB, which is usually increased in cancer cells. Therefore, 9AA is a compound with two very important activities: it inhibits NF-κB (usually overactive in cancer) and activates p53 (usually inhibited in cancer). Although 9AA possesses several very important properties as a candidate anti-cancer agent, associated toxicities made its further development unattractive.

### SUMMARY OF THE INVENTION

The present invention provides a method of treating cancer by administering to a subject in need thereof a therapeutically effective amount of a tyrosine kinase inhibitor and an NF-κB inhibitor. In some embodiments, the cancer includes an EGFR activating mutation, while in further embodiments the subject has cancer that has developed resistance to tyrosine kinase inhibitors. The resistance can result from various changes. For example, the resistance can result from second site mutation of the EGFR receptor, or from mutated KRAS in tumors with wildtype EGFR receptor or MET amplification. In further embodiments, the cancer is non-small cell lung cancer.

A variety of different tyrosine kinase inhibitors and NF-kB inhibitors can be used. In some embodiments, the tyrosine kinase inhibitor is erlotinib or gefitnib. In additional embodiments, the NF-κB inhibitor is a curaxin or a quinacrine derivative (e.g., quinacrine). The tyrosine kinase inhibitors can be administered proximately in time, and in some embodiments they can be administered simultaneously. In further embodiments, the tyrosine kinase inhibitor and an NF-κB inhibitor are both administered in a pharmaceutically acceptable carrier.

### BRIEF DESCRIPTION OF THE FIGURES

The present invention may be more readily understood by reference to the following drawings, wherein:

FIG. 1 provides illustrations showing the role of tyrosine kinase mutation in cancer development (1A), and the role of further mutation in the development of resistance to tyrosine kinase inhibitors (1B).

FIG. 2 provides an illustrative scheme showing the proposed mechanism of curaxins and quinacrine derivatives for inhibiting NF-κB.

FIG. 3 provides dose-response curves of various cell lines to a combination of erlotinib and quinacrine.

FIG. 4 provides a graph showing synergy between erlotinib and quinacrine as quantified with the CalcuSyn software using the Chou-Talalay method.

FIG. 5 provides an image and graph showing that addition of quinacrine to erlotinib treatment inhibited colony formation.

FIG. 6 provides images of staining on flow cytometric analysis showing that quinacrine induces apoptosis in erlotinib-resistant NSCLC cells.

FIG. 7 provides a Western Blot image of PARP cleavage showing that quinacrine induces apoptosis in erlotinib-resistant NSCLC cells.

FIG. 8 provides graphs of cell cycle analysis showing that erlotinib plus quinacrine induces G1/S cell cycle arrest in A549 NSCLC cells.

FIG. 9 provides graphs of cell cycle analysis showing that erlotinib plus quinacrine indices G1/S cell cycle arrest in H1975 NSCLC cells.

FIG. 10 provides graphs quantifying the G1/S and G2/M cell cycle arrest induced by erlotinib plus quinacrine.

FIG. 11 provides graphs showing that quinacrine but not chloroquine suppresses NF-κB-dependent luciferase reporter activity.

FIG. 12 provides graphs showing that quinacrine but not chloroquine suppresses IL-1-induced NF-κB-dependent luciferase reporting activity.

FIG. 13 provides Western blot images showing that quinacrine but not chloroquine inhibits SSRP1, a FACT subunit.

FIG. 14 provides a graph showing that the loss of shSSRP1 decreases cell viability in H1975 cells.

FIG. 15 provides a graph showing that the loss of shSSRP1 decreases colony formation in A549 cells.

FIG. 16 provides graphs showing that SSRP1 knockdown sensitizes NSCLC cells to erlotinib treatment.

FIG. 17 provides graphs showing that EGFR/HER2 dual TKI lapatinib and NF-κB inhibitor CBL0137 are synergistic in stem and non-stem glioblastoma multiforme (GBM) cells.

### DETAILED DESCRIPTION OF THE INVENTION

The present invention provides a method of treating cancer by administering to a subject in need thereof a therapeutically effective amount of a tyrosine kinase inhibitor (TKI) and an NF-κB inhibitor. The method is useful for treating subjects having cancer that has developed resistance to tyrosine kinase inhibitors. In some embodiments, for example where the TKI is erlotinib and the NF-κB inhibitor is quinacrine, the combined agents provide a synergistic antitumor effect.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for thiazolidinediones of this invention are those that do not interfere with the energy restriction activity of the thiazolidinediones. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups.

Cycloalkyl, as used herein, refers to an alkyl group (i.e., an alkyl, alkenyl, or alkynyl group) that forms a ring structure. Cyclic groups can be monocyclic or polycyclic and preferably have from 3 to 10 ring carbon atoms. A cycloalkyl group can be attached to the main structure via an alkyl group including 4 or less carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halo moieties include chlorine, bromine, fluorine, and iodine.

The term “aryl” as used herein includes carbocyclic aromatic rings or ring systems. Examples of aryl groups include phenyl, naphthyl, biphenyl, fluorenyl and indenyl. Aryl groups may be substituted or unsubstituted.

Unless otherwise indicated, the term “heteroatom” refers to the atoms O, S, or N. The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The invention is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc. The subject may be at risk due to exposure to carcinogenic agents, being genetically predisposed to disorders characterized by unwanted, rapid cell proliferation, and so on.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of improvement in disease severity and the frequency of incidence over treatment of each agent by itself, while avoiding adverse side effects typically associated with alternative therapies.

### Cancer Treatment

A method of treating cancer is described herein. The method includes administering a therapeutically effective amount of a tyrosine kinase inhibitor (TKI) and an NF-κB inhibitor to the subject in need thereof.

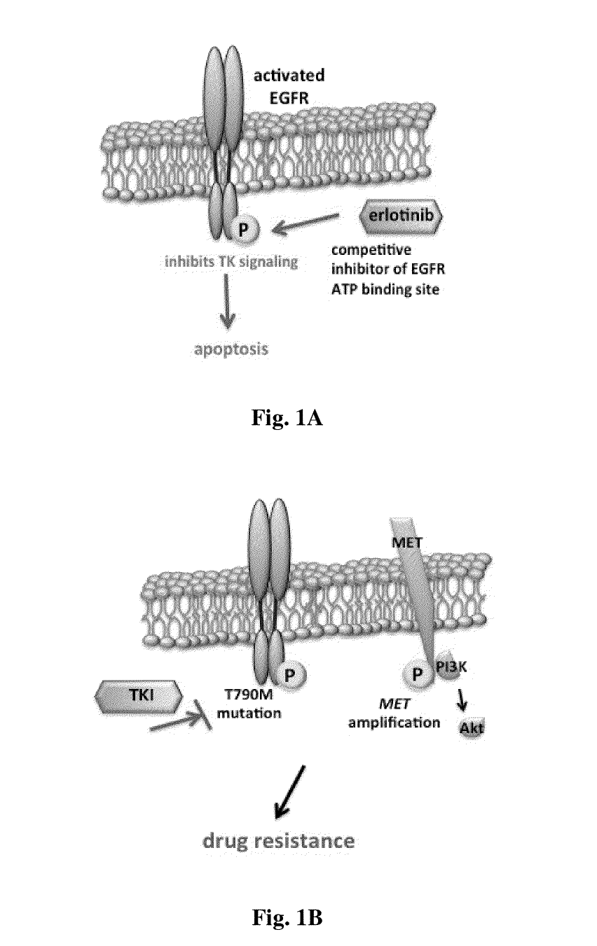

Subjects with cancer typically develop resistance to treatment with tyrosine kinase inhibitors through further mutation of EGFR or through activation of downstream survival pathways via MET amplification, as shown in FIGS. 1A and 1B. Mutations in the epidermal growth factor receptor activate signaling pathways that promote cell survival. Accordingly, some embodiments of the invention are directed to treating cancer that includes an EGFR activating mutation. Examples of these mutations include in-frame exon 19 deletion, and L858R substitution in exon 21 in NSCLC. Cancer cells such as NSCLC cells become dependent on these survival signals. Downstream activation of antiapoptotic signals (via PI3K/Akt) is greatly enhanced in cells harboring mutated EGFR compared to wild type, (range 30%-100%, with most series reporting response rates >60%). TKIs provide a selective anti-proliferative effect by binding with a higher affinity to these mutant receptors. As a result EGFR-mut(+) NSCLC patients have a rapid and often dramatic clinical response to TKIs. See Lynch et al, N Engl J Med, 350: 2129-39 (2004). In glioblastomas, coexpression of the EGFR deletion mutant variant III (EGFRvIII) and PTEN is associated with responsiveness to EGFR kinase inhibitors. See Mellinghoff et al, N Engl J Med, 353: 2012-24 (2005).

FIG. 1B shows how cancer cells develop resistance to TK inhibitors through further mutation of the EGFR. Subjects eventually develop resistance to TKIs, with a medium time to the development of resistance being 10-14 months. The efficacy of TKIs (e.g., erlotinib) is limited by either primary or secondary resistance. For example, resistance may result from a second site mutation (e.g., the second-site mutation EGFR-L858R/T790M) of the EGFR receptor in initially sensitive patients. Alternately, resistance may result from mutated KRAS in tumor with wild-type EGFR, or in tumors that develop or Met amplification.

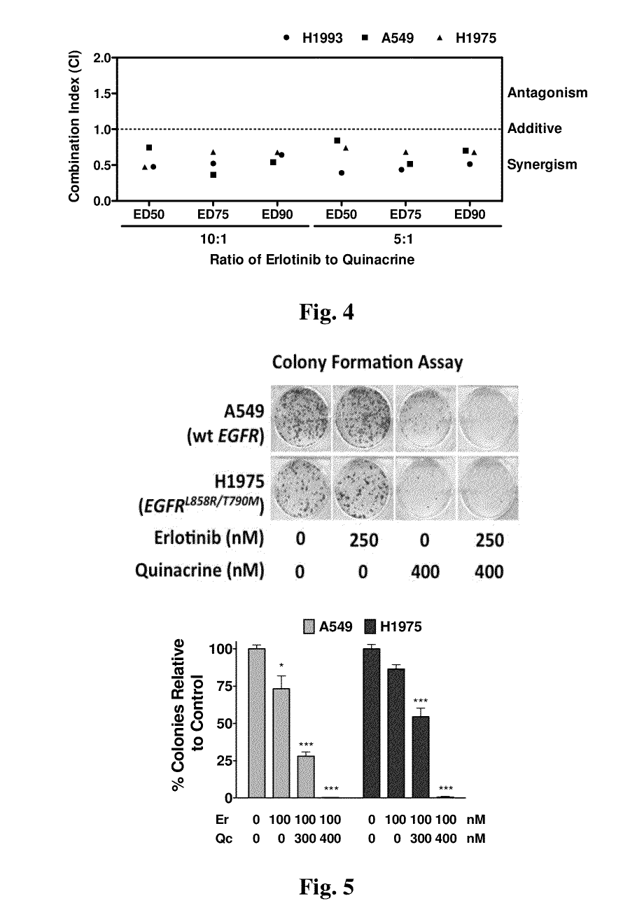

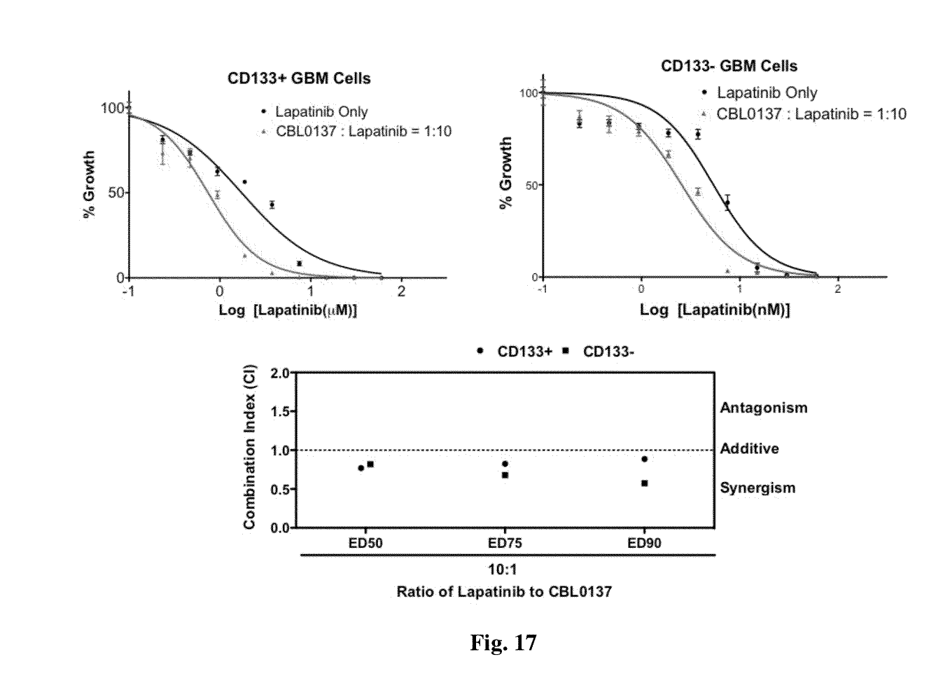

The present invention provides a method to overcome the resistance to TKI anticancer agents by also administering an NF-κB inhibitor to the subjects receiving the TKI. While not intending to be bound by theory, NF-κB activation appears to be involved in the development of resistance to TKIs and other chemotherapeutic agents, and therefore this resistance can be overcome by inhibiting NF-κB activation. The combination of the TKI and the NF-κB inhibitor provides a synergistic anticancer effect that overcomes the TKI resistance that has developed. A synergistic effect, as defined herein, is an anticancer effect that is more than the additive anticancer effect that the two agents would provide if used in isolation. The Chou-Talalay method quantifies the effects of drug combination by the combination index (CI): CI=1 for additive effect, CI<1 for synergism, and CI>1 for antagonism. See Chou, Cancer Res, 70: 440-6 (2010). FIG. 4 shows the CI for the combination of erlotinib and quinacrine, a TKI and an NF-κB inhibitor respectively, showing that the combination is synergistic in NSCLC. FIG. 17 shows the CI for the combination of lapatinib and CBL0137, also a TKI and an NF-κB inhibitor respectively, showing that the combination is synergistic in GBM.

A wide variety of tyrosine kinase inhibitors are known and suitable for use in the method. Tyrosine kinases are enzymes responsible for the activation of many proteins by signal transduction cascades, and, mutation of the tyrosine kinases resulting in their increased activation can be a significant factor in tumor development. For example, a number of small molecule EGFR TKI drugs have been developed for treating non-small cell lung cancer, including erlotinib, gefitinib, afatinib, icotinib, NOV120101, BMS-690514, CO-1686, HM61713, dacomitinib, CUDC-101, AP26113, and XL647. See Berardi et al., Onco Targets Ther. 6:563-76 (2013), the disclosure of which is incorporated herein by reference. In addition, a number of VEGF TKI drugs have been developed for the treatment of renal cell carcinoma, including axitinib, tivozanib, sunitinib, and pazobanib. See Bukowski, R. M., Front Oncol., 2:13 (2012), the disclosure of which is incorporated by reference. Several TKIs targeting EGFR have been used in patients with malignant gliomas, among which 50-60% have EGFR gene overexpression and 24-67% of cases express the EGFR mutant EGFRVIII, with mixed results. See Ye et al, Expert Opin Ther Targets, 14: 303-16 (2010). For a general reference for the use of TKIs in cancer, see Zhang et al, Nat Rev Cancer 9: 28-39 (2009).

In some embodiments, the tyrosine kinase inhibitor erlotinib can be used. When used for monotherapy of NSCLC, eroltinib is administered daily at a dose of 150 mg. When combined with gemcitabine for treatment of pancreatic cancer, erlotinib is administered daily at a dose of 100 mg. Erolotinib targets the human epidermal growth factor receptor pathway (HER1 or EGFR). Erlotinib has a half life of about 36 hours and is cleared predominantly by CYP3A4 methabolism. Erlotinib has the structure shown in Formula I below:

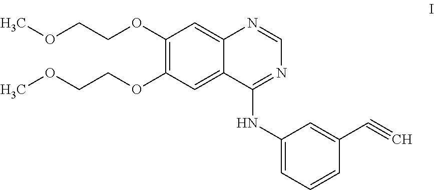 [SMILES: C#Cc1cccc(Nc2ncnc3cc(OCCOC)c(OCCOC)cc23)c1]

The method of the invention also includes administration of an NF-κB inhibitor. NF-κB (nuclear factor kappa-light-chain-enhancer of activated B cells) is a protein complex that controls the transcription of DNA, and dysregulation of NF-κB is associated with cancer. A large number of compounds have been identified as being NF-κB inhibitors. These include 17-AAG, TMFC, AQC derivatives, 9-aminoacridine derivatives, chromene derivatives, curaxins, D609, dimethylfumarate, EMDPC, histidine, HIV-1 PI, mesalamine, PEITC, pranlukast, RO31-8220, SB203580, tetrathiomolybdate, diferoxamine, dihydroisoeugenol, dihydrolipoic acid, dilazep, fenofibric acid, DMDTC, dimethylsulfoxide, disulfiram, ebselen, edaravone, EGTA, EPC-K1, epigallocatechin-3-gallate, erogthioneine, ethyl pyruvate, garcinol, metatein, hudroquinone, IRFI 042, iron tetrakis, isovitexein, kangen-karyu extract, ketamine, lacidipine, lazaroids, L-cysteine, adiponectin, pioglitazone, perfenidone, quinadrin, tranilast, troglitazone, and quinacrine derivatives. See Gupta et al., Biochim Biophys Acta, 1799, 775-787 (2010), the disclosure of which is incorporated herein by reference.

In some embodiments, the NF-κB inhibitor is a curaxin or a quinacrine derivative. Curaxins are a set of small molecule anticancer agents based on 9-aminoacridine that were identified by structure-activity studies as having anticancer activity. See Gasparian et al., Sci Transl Med, 3:95 (2011), the disclosure of which is incorporated herein by reference. Examples of particularly active curaxins are shown below as formula IIa-c, with IIa being designated CBLC000, IIb being designated CBLC100, and IIc being designated CBLC137.

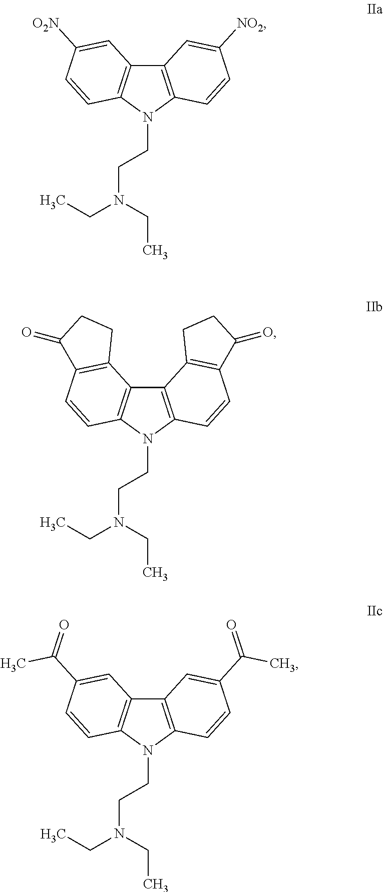 [SMILES: CCN(CC)CCn1c2ccc([N+](=O)[O-])cc2c2cc([N+](=O)[O-])ccc21 ; CCN(CC)CCn1c2ccc3c(c2c2c4c(ccc21)C(=O)CC4)CCC3=O ; CCN(CC)CCn1c2ccc(C(C)=O)cc2c2cc(C(C)=O)ccc21]

The NF-κB inhibitor can also be a quinacrine derivative. A quinacrine derivative, as defined herein, is a structure according to formula III:

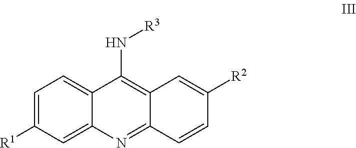

wherein R¹ is a H, Me, or halogen, R² is H, Me, OH, or OMe, and R³ is C₄-C₁₂ alkyl or alkylamino group.

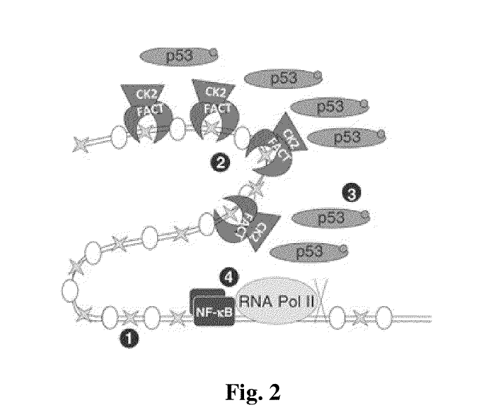

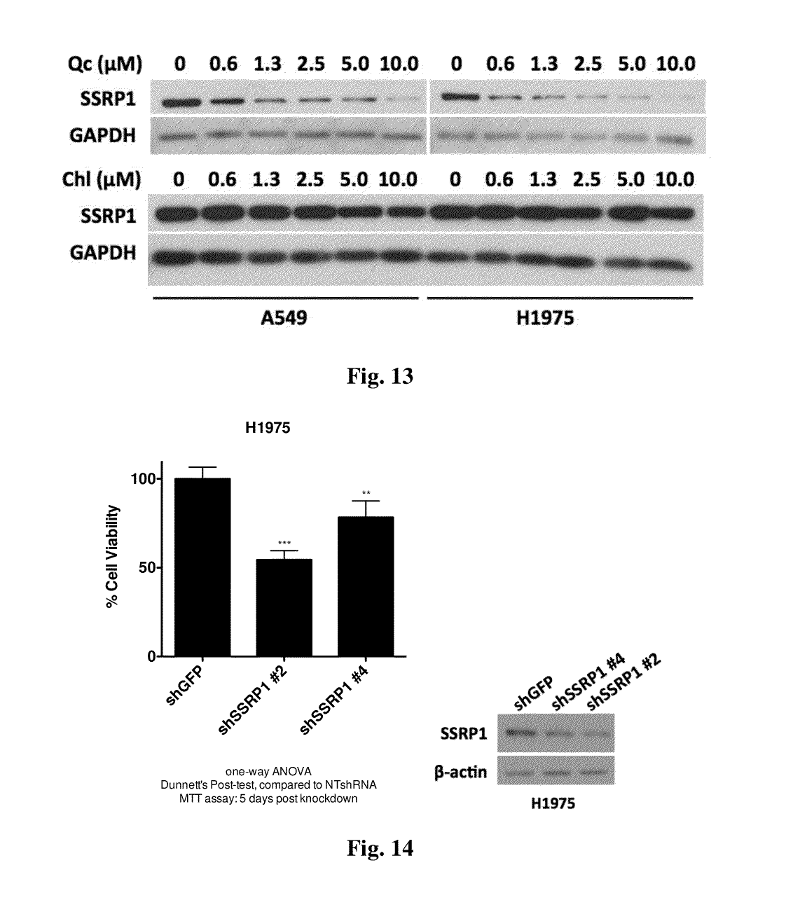

Curaxins and quinacrine derivatives intercalate with DNA with the planar aryl ring, while the “tail” of the molecule extends into the DNA minor groove. While not intending to be bound by theory, it is believed that curaxins and quinacrine derivatives suppress NF-κB by causing chromatin trapping of the FACT (facilitates chromatin transcription) complex. See Gasparian et al., Sci Transl Med, 3:95 (2011). The FACT complex is a heterodimer including the structure specific recognition protein (SSRP1) and suppressor of Ty16 (SPT16). Its normal function is to promote replication of fork progression by disassembling nucleosomes in front of RNA polymerase II during transcription elongation. However, FACT is often expressed in aggressive, undifferentiated cancers, and neoplastic (but not normal) cell growth depends on FACT activity. See Garcia et al. Cell Reports 4, 159-173 (2013). As shown in FIG. 2, curaxins (or quinacrine derivatives) bind to DNA and disturb chromatin architecture so that FACT becomes “trapped.” This results in activating phosphorylation of p53 by FACT-associated CK2, and reduced NF-κB transcription because of the depletion of soluble FACT. FIG. 13 shows that quinacrine treatment inhibits FACT (SSRP1) in A549 and H1975 NSCLC cells.

In other embodiments, the NF-κB inhibitor is quinacrine, which has the structure shown in formula IV:

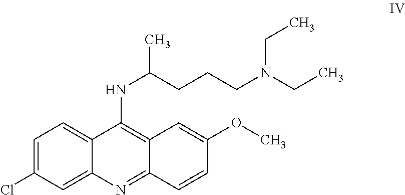 [SMILES: CCN(CC)CCCC(C)Nc1c2ccc(Cl)cc2nc2ccc(OC)cc12]

Quinacrine was identified during studies of 9-aminoacridine-related compounds as having the ability to activate p53 and inhibit NF-κB. See Gurova et al., Proc. Natl Acad. Sci. U.S.A. 102, 17448-17453 (2005). Quinacrine demonstrated an anti-tumor effect in mice against xenograft tumors originating from human renal cell carcinoma, hand has activity against a number of other different tumor cell lines, such as prostate cancer cells. Quinacrine is administered in dosages ranging from 100 mg to 1000 mg a day when used to treat malaria, and has a half life that averages from 7 to 10 days.

TKI and NF-κB inhibitors can be used to both treat and prevent cancer. As used herein, the term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those at risk of developing precancers and cancers.

Cancer cells, as defined herein, are cells that contain genetic damage that has resulted in the relatively unrestrained growth of the cells. The genetic damage present in a cancer cell is maintained as a heritable trait in subsequent generations of the cancer cell line. The cancer treated by the method of the invention may be any of the forms of cancer known to those skilled in the art or described herein. Cancer that manifests as both solid tumors and cancer that instead forms non-solid tumors as typically seen in leukemia can be treated. Based on the prevalence of an increase in aerobic glycolysis in all types of cancer, the present invention provide methods for treating a subject that is afflicted with various different types of cancers, including carcinoma, sarcoma, and lymphoma. Examples of types of cancer that can be treated using the compounds of the invention include ovary, colon, lung, breast, thyroid, and prostate cancer, while additional embodiments are directed to only prostate cancer, breast cancer, and pancreatic cancer. For the present invention, some embodiments are directed to the treatment of tyrosine-kinase dependent cancers. In some embodiments, the cancer is non-small cell lung cancer.

The effectiveness of cancer treatment may be measured by evaluating a reduction in tumor load or decrease in tumor growth in a subject in response to the administration of the TKI and NF-κB inhibitors. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

A subject, as defined herein, is an animal, preferably a mammal such as a domesticated farm animal (e.g., cow, horse, pig) or a pet (e.g., dog, cat). More preferably, the subject is a human. The subject may also be a subject in need of cancer treatment. A subject in need of cancer treatment can be a subject who has been diagnosed as having a disorder characterized by unwanted, rapid cell proliferation. Such disorders include, but are not limited to cancers and precancerous conditions.

Because the NF-κB inhibitor derives a significant portion of its effectiveness from its ability to overcome resistance to TKI by the cancer cells, the TKI and the NF-κB inhibitor should be administered close enough together in time for the NF-κB inhibitor to increase the TKI's anticancer effect, which is referred to herein as being administered proximately in time. What constitutes proximately in time can vary with the metabolism of the individual, and the dose of the TKI and/or NF-κB inhibitor administered. In some embodiments, proximate in time can be within 1 hour, within 6 hours, within 12 hours, or within 24 hours of administration of the other agent. In some embodiments, the TKI and the NF-κB inhibitor are administered simultaneously. However, in other embodiments, the NF-κB inhibitor can be administered proximately in time either before or after TKI administration, or proximately in time before TKI administration, or proximately in time after TKI administration.

The TKI and NF-κB inhibitors may be administered alone or in conjunction with other antineoplastic agents or other growth inhibiting agents or other drugs or nutrients, as in an adjunct therapy. The phrase “adjunct therapy” or “combination therapy” in defining use of a compound described herein and one or more other pharmaceutical agents, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

For the purposes of combination therapy, there are large numbers of antineoplastic agents available in commercial use, in clinical evaluation and in pre-clinical development, which could be selected for treatment of cancers or other disorders characterized by rapid proliferation of cells by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents. Alternatively, other anti-neoplastic agents, such as metallomatrix proteases inhibitors (MMP), such as MMP-13 inhibitors, or αᵥβ₃ inhibitors may be used. Suitable agents which may be used in combination therapy will be recognized by those of skill in the art. Similarly, when combination with radiotherapy is desired, radioprotective agents known to those of skill in the art may also be used. Treatment using compounds of the present invention can also be combined with treatments such as hormonal therapy, proton therapy, cryosurgery, and high intensity focused ultrasound (HIFU), depending on the clinical scenario and desired outcome.

Candidate agents (e.g., tyrosine kinase or NF-κB inhibitors) may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof.

Administration and Formulation of TKI and NF-κB inhibitors

The present invention provides a method for administering one or more thiazolidinedione derivatives in a pharmaceutical composition. Examples of pharmaceutical compositions include those for oral, intravenous, intramuscular, subcutaneous, or intraperitoneal administration, or any other route known to those skilled in the art, and generally involves providing the thiazolidinedione derivative formulated together with a pharmaceutically acceptable carrier.

When preparing the compounds described herein for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

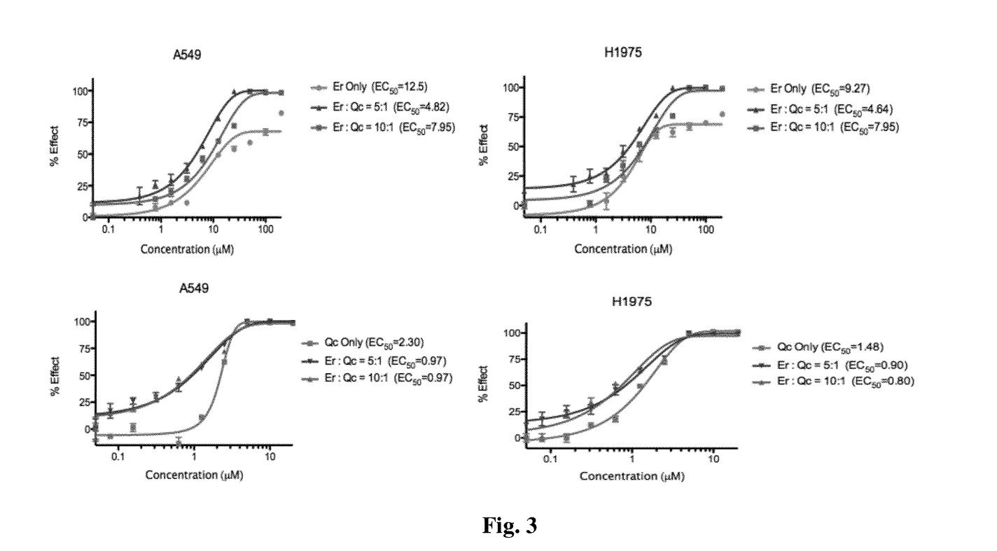

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. The pharmaceutical compositions may contain active ingredient in the range of about 0.1 to 2000 mg, preferably in the range of about 0.5 to 500 mg and most preferably between about 1 and 200 mg. A daily dose of about 0.1 to 20 mg/kg body weight, preferably between about 1.0 and about 10 mg/kg body weight, may be appropriate. The daily dose can be administered in one to four doses per day. Based on the IC₅₀ values of erlotinib and quinacrine in NSCLC cell lines, the inventors have determined that a 1:5 and 1:10 ratio of quinacrine to erlotinib is highly synergistic in erlotinib-resistant NSCLC cells. See FIGS. 3 and 4.

For example, the maximum tolerated dose (MTD) for TKI and NF-κB inhibitors can be determined in tumor-free athymic nude mice. Agents are prepared as suspensions in sterile water containing 0.5% methylcellulose (w/v) and 0.1% Tween 80 (v/v) and administered to mice (7 animals/group) by oral gavage at doses of 0, 5, 10 and 20 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

The TKI and NF-κB inhibitors can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of compounds of formula I may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Synergistic Combination of Quinacrine and Erlotinib in Erlotinib-Resistant Non-Small Cell Lung Cancer (NSCLC) Cells

### Introduction

Erlotinib is an epidermal growth factor receptor (EGFR) tyrosine kinase inhibitor (TKI). It is highly effective in 10-30% of NSCLC patients harboring somatic, activating mutation sof EGFR (most commonly exon 19 del or exon 21 L858R mutation). However, all patients develop secondary resistance to erlotinib in 8-12 months. The inventors believe nuclear factor kappa B (NF-κB) activation is an important mechanism of survival and resistance for these NSCLC tumors. The antimalarial drug quinacrine belongs to a class of 9-aminoacridine (9-AA)-derived compounds. To improve treatment with erlotinib, the inventors combined it with quinacrine in several erlotinib-resistant non-small cell lung adenocarcinoma cells. The inventors have found that quinacrine profoundly suppresses NF-κB activity and selectively kills cancer cells but not normal cells.

### Methods & Results

Erlotinib-resistance NSCLC cell lines A549, H1975, and H1993 were used. A549 cells harbor wild-type EGFR and mutant KRAS (G61H), H1975 cells have activating EGFR^{L858R} as well as the second site T790M EGFR mutation, and H1993 cells have wild-type EGFR and c-met amplification. These cell lines therefore represent three major mechanisms of resistance to erlotinib in NSCLC patients.

In three erlotinib-resistant NSCLC cell lines (A549, H1975, and H1993) the inventors tested the effect of single and combination treatment of erlotinib and quinacrine on cell survival with MTT assay. The cells were plated in 96-well plates at 2500-3000 cells per well, and allowed to attach overnight, with 6 replicates being used for each dose. First, the inventors detected the IC₅₀ with single drug treatment for each cell line. The IC₅₀ for erlotinib is around 20 nm in the sensitive cell lines and 10-20 μM in the resistant cell lines. The IC₅₀ for quinacrine is around 1-2 μM in all cell lines. Based on the IC₅₀ ratios of the two drugs, the inventors treated the resistant cell lines with 5:1 or 10:1 ratio of erlotinib to quinacrine. Cell survival is accessed after 72 hours of drug treatment using the MTT assay. The results can be seen in FIG. 3. Drug synergy is quantified with the CalcuSyn software using the Cou-Talalay method, as shown in FIG. 4. Chou T C, Cancer Res. 70, 440-6 (2010). In A549 (wtEGFR), H1975 (EGFR-L858R/T790M) and H1993 (Met amplification) cells, the combination of erlotinib and quinacrine at 5 to 1 or 10 to 1 fixed ratios was highly synergistic, as quantified by the Chou-Talalay combination indices [ED50: 0.61 (0.42-0.81); ED75: 0.53 (0.40-0.67); ED90: 0.63 (0.54-0.71)].

FIG. 5 provides the results of a clonogenic assay showing that addition of quinacrine to erlotinib treatment inhibits colony formation. For colony formation assay, cells were seeded in 6-well plates at 500 per well, allowed to attach overnight, and treated with 1 μM of erlotinib, 3 μM or 5 μM quinacrine or a combination of both in triplicates. Drugs were replaced every 72 hours. After 14 days, cells were fixed with 100% methanol and stained with 1% crystal violet. Colonies were quantified using the cell counter plugin of the NIH ImageJ software (v.1.46). The highest level of colony formation inhibition was achieved when both erlotinib (250 nM) and quinacrine (400 nM) were used.

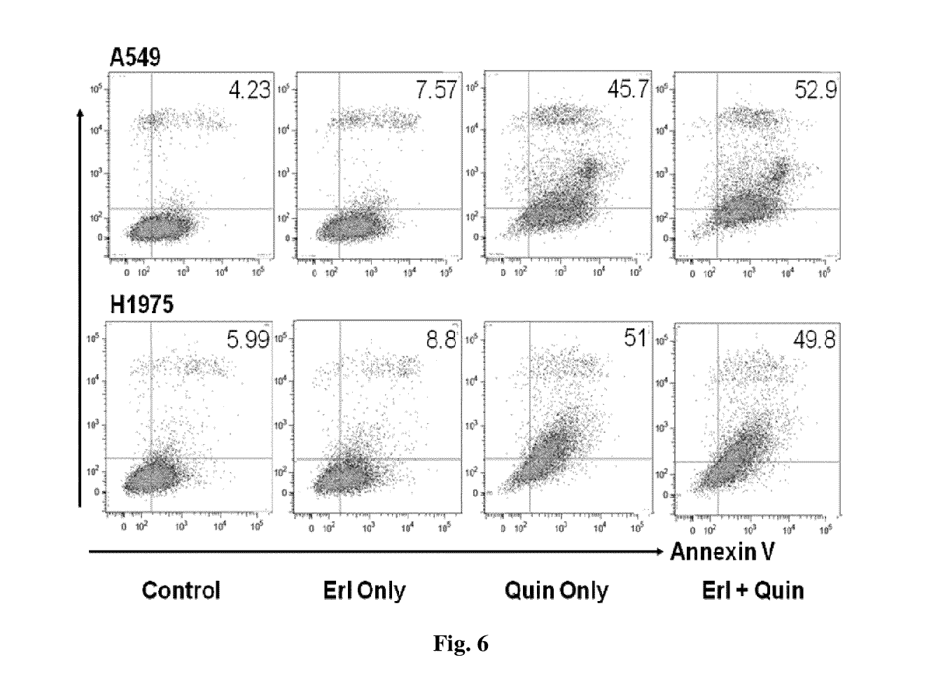

FIG. 6 provides images of staining on flow cytometric analysis showing that quinacrine induces apoptosis in Erlotinib-resistance NSCLC cells. Addition of quinacrine to erlotinib treatment induced significant apoptosis in A549 and H1975 cells after 48h of drug treatment, as shown by Annexin V-PI staining on flow cytometry analysis. Analysis of apoptosis. Annexin V staining was performed using Annexin V-APC (eBioscience, #88-8007) in conjunction with propidium iodide staining according to manufacturer's protocol, and assessed by FACScan.

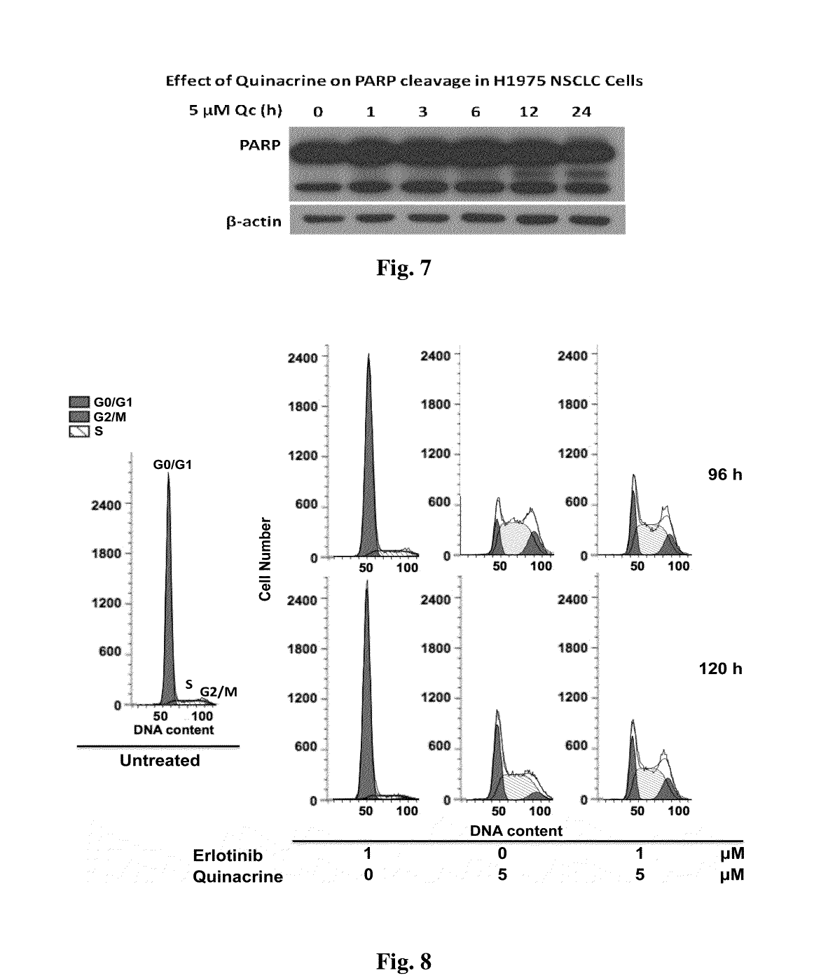

FIG. 7 shows Western blots that show that quinacrine induces apoptosis in Erlotinib-Resistant NSCLC cells. Quinacrine induced significant apoptosis in H1975 cells, as shown by time-dependent increase of PARP cleavage. H1975 cells were treated with 5 μM quinacrine for the indicated time-points. Western blotting was used to detect PARP cleavage as an indicator of apoptosis.

FIG. 8 provides graphs of cell cycle analysis showing that erlotinib plus quinacrine induces G1/S cell cycle arrest in A549 NSCLC cells. Addition of quinacrine to erlotinib treatment induced significant G1/S and G2/M cell cycle arrest in A549 cells. A549 cells were treated with 1 μM of erlotinib, 3 μM or 5 μM of quinacrine or a combination of both for 96 hours or 120 hours, and were then fixed with 100% cold ethanol at −20° C. for 1 hour to overnight, and stained with 3 μM of propidium iodide (Invitrogen, #P3566) in the presence of RNase for 15 min at room temperature. Cell cycle distribution was assessed by FACScan (BD Biosciences, San Jose, Calif.) analysis.

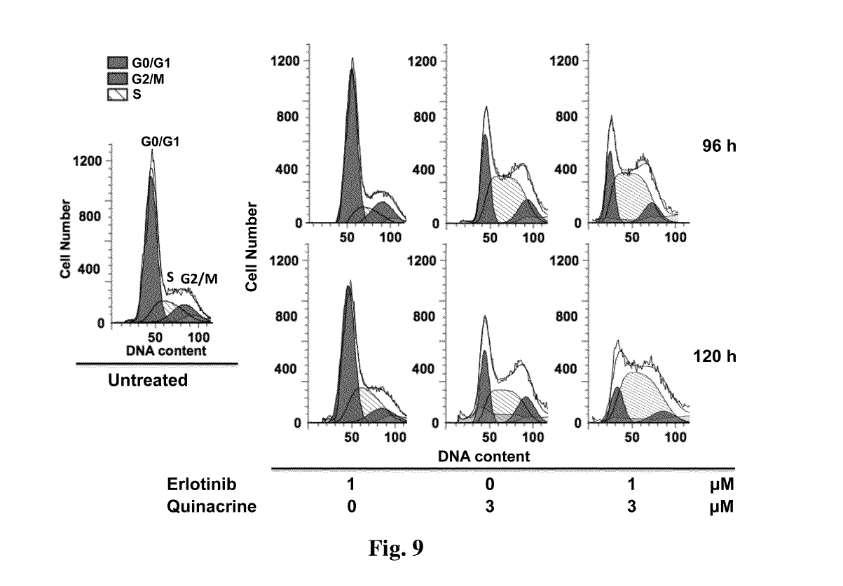

FIG. 9 provides graphs showing that erlotinib plus quinacrine induces G1/S cell cycle arrest in H1975 NSCLC cells. Addition of quinacrine to erlotinib treatment induced significant G1/S and G2/M cell cycle arrest in H1975 cells. Cell-Cycle Analysis. H1975 cells were treated with 1 μM of erlotinib, 3 μM or 5 μM of quinacrine or a combination of both for 96 hours or 120 hours, and were then fixed with 100% cold ethanol at −20° C. for 1 hour to overnight, and stained with 3 μM of propidium iodide (Invitrogen, #P3566) in the presence of RNase for 15 min at room temperature. Cell cycle distribution was assessed by FACScan (BD Biosciences, San Jose, Calif.) analysis.

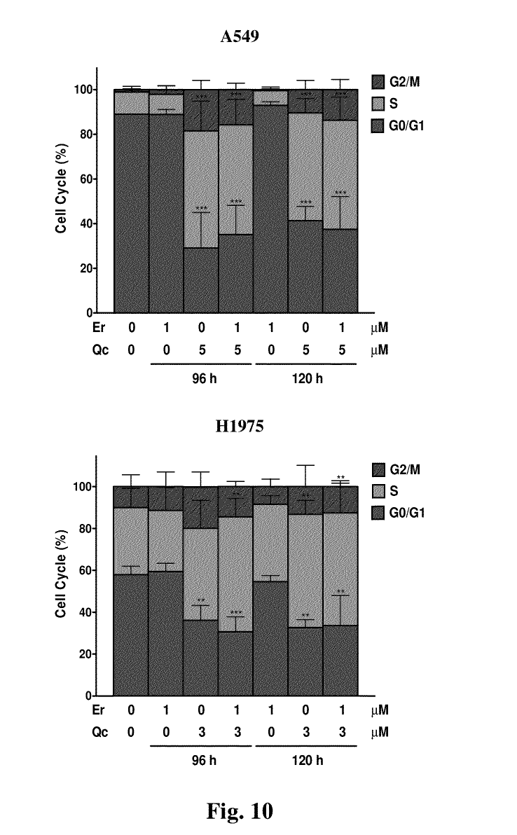

FIG. 10 provides graphs showing that erlotinib plus quinacrine induces G1/S cell cycle arrest in NSCLC cells. Quantification of G1/S and G2/M cell cycle arrest from the flow cytometry histogram data in A549 and H1975 cells. Experiment was repeated 3 times. Statistical anlaysis indicates that addition of quinacrine to erlotinib treatment induced significant G1/S and G2/M cell cycle arrest in both cell lines.

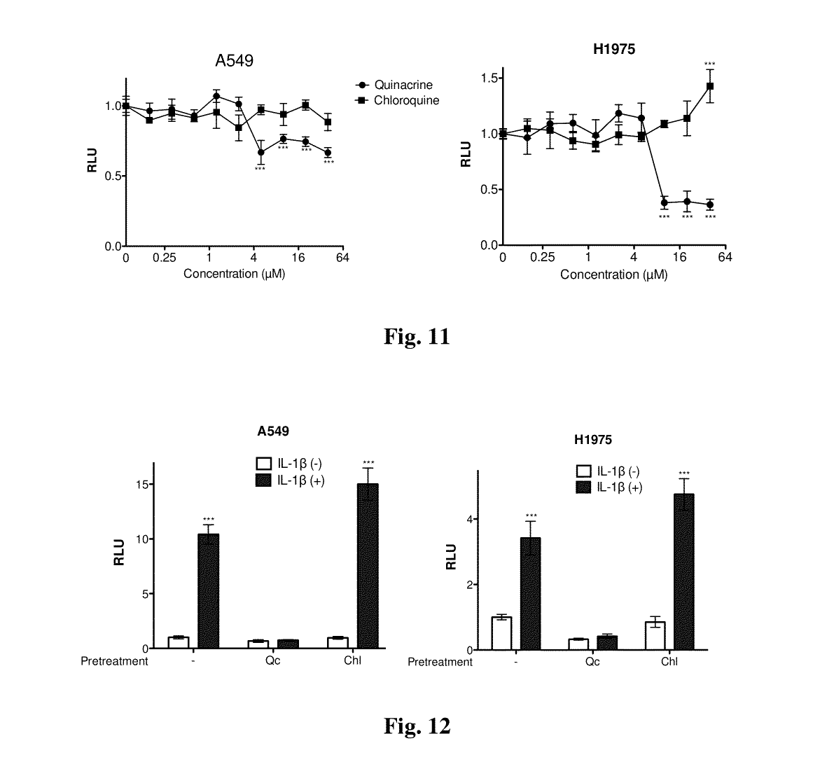

FIG. 11 provides graphs showing that quinacrine but not chloroquine suppresses NF-κB-dependent luciferase reporter activity. Relative luciferase unit (compared to untreated control) was quantified in A549 or H1975 cells stably expressing NF-κB luciferase reporter after 4 h of quinacrine treatment. To ensure that this decrease is not due to decrease in cell viability, this time point is chosen when no significant change in cell viability was found by MTT assay. For the NF-κB luciferase assay, A549 or H1975 cells were infected with the κB-luciferse construct PLANFκBluc and stably selected with puromycin or hygromycin. The reporter cells were then seeded in 96-well plates at 1-2×10³ per well, allowed to attach overnight, and then treated with drugs and/or interleukin-1 for four hours. Cells were then harvested in reporter lysis buffer (Promega) and assayed for luciferase activity using the luciferase assay system (Promega).

FIG. 12 provides graphs showing that quinacrine but not chloroquine suppresses interleukin-1 (IL-1)-induced NF-κB-dependent luciferase reporting activity. Relative luciferase unit (compared to untreated control) was quantified in A549 or H1975 stable NF-κB luciferase reporter cells pretreated with quinacrine or chloroquine, and then stimulated with interleukin-1 for 6 h. For the NF-κB luciferase assay, A549 or H1975 cells were infected with the κB-luciferse construct PLANFκBluc and stably selected with puromycin or hygromycin. The reporter cells were then seeded in 96-well plates at 1-2×10³ per well, allowed to attach overnight, and then treated with drugs and/or IL-1. Cells were then harvested in reporter lysis buffer (Promega) and assayed for luciferase activity using the luciferase assay system (Promega).

FIG. 13 provides Western blot images showing that quinacrine but not chloroquine inhibits FACT. Upon quinacrine treatment, the FACT subunit SSRP1 disappeared from the soluble protein fraction, indicating that FACT is “trapped” in the insoluble nuclear pellet by quinacrine treatment. Soluble protein fractions were prepared by incubating cell pellets with occasional vortexing in lysis buffer containing 50 mM Tris (pH 8.0), 150 mM NaCl, 1.0% NP-40 with protease inhibitors (10 μg/ml aprotinin, 5 μg/ml leupeptin,1 mM phenylmethane sulfonl fluoride) and then centrifuged at 14,000 rpm for 10 min, discarding the crude nuclear pellet. Cell extracts containing equal quantities of proteins, determined by the Bradford method, were separated by 10% SDS-PAGE and transferred to polyvinylidene difluoride membranes (Millipore). Primary antibodies against SSRP1 (BioLegend) were detected with goat anti-mouse conjugated to horseradish peroxidase (Rockland), using enhanced chemiluminescence (Perkin-Elmer).

FIG. 14 provides a graph showing that the loss of shSSRP1 decreases cell viability in H1975 cells. H1975 cells were infected with shRNA against GFP or SSRP1 and then selected with puromycin. Cells are plated in quadruplicates in 96-well plate and cell viability was measured by MTT assay. To carry out the shRNA-mediated knockdown, Lentiviral plasmids encoding shRNAs targeting GFP or SSRP1 from Sigma-Aldrich (TRCN0000019270, “#2”; TRCN0000019272, “#4”) were kindly gifted by Dr. Katerina Gurova. Viruses were packaged in 293T cells using the second-generation packaging constructs pCMV-dR8.74 and pMD2G. Supernatants containing virus were collected at 48 hours and supplemented with 1 μg/ml polybrene before being used to infect cells for 6 hours. Knockdown efficiency was evaluated by Western blotting 48 hours post infection.

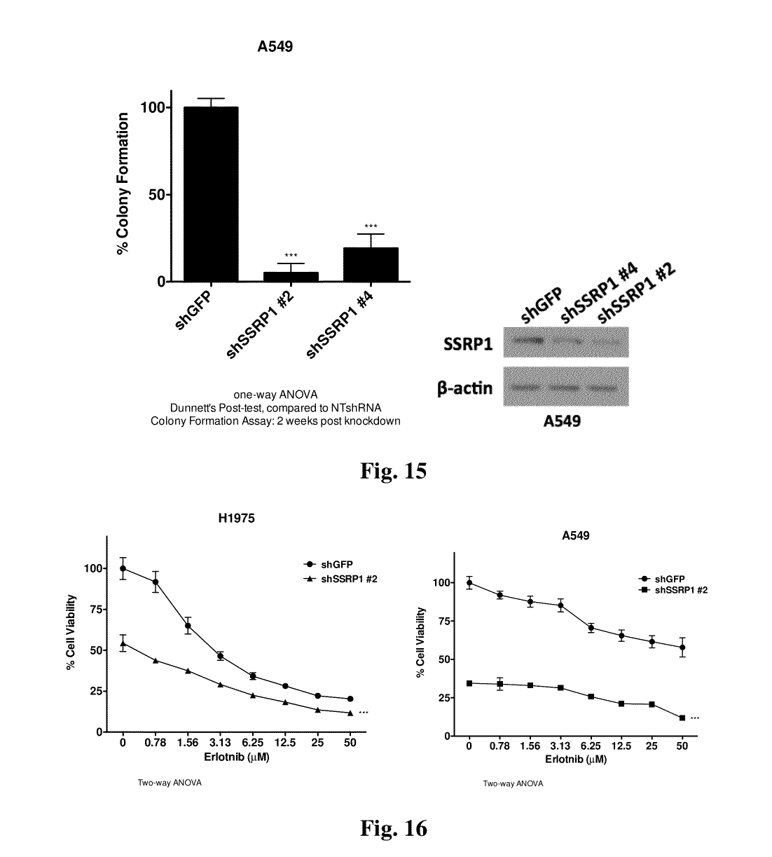

FIG. 15 provides a graph showing that the loss of shSSRP1 decreases colony formation in A549 cells. A549 cells were infected with shRNA against GFP or SSRP1 and then selected with puromycin. Cells were plated in 6-well plates in triplicates at 500 cells/well and cell colonies were quantified after 2 weeks by crystal violet staining. To carry out the shRNA-mediated knockdown, Lentiviral plasmids encoding shRNAs targeting GFP or SSRP1 from Sigma-Aldrich (TRCN0000019270, “#2”; TRCN0000019272, “#4”) were kindly gifted by Dr. Katerina Gurova. Viruses were packaged in 293T cells using the second-generation packaging constructs pCMV-dR8.74 and pMD2G. Supernatants containing virus were collected at 48 hours and supplemented with 1 μg/ml polybrene before being used to infect cells for 6 hours. Knockdown efficiency was evaluated by Western blotting 48 hours post infection.

FIG. 16 provides graphs showing that SSRP1 knockdown sensitizes NSCLC cells to erlotinib treatment. A549 or H1975 cells were infected with shSSRP1 and plated in 96-well plates and treated with increasing concentrations of Erlotinib over 72 h in quadruplicates. Cell viability was measured by MTT assay. To carry out the shRNA-mediated knockdown, Lentiviral plasmids encoding shRNAs targeting GFP or SSRP1 from Sigma-Aldrich (TRCN0000019270, “#2”; TRCN0000019272, “#4”) were kindly gifted by Dr. Katerina Gurova. Viruses were packaged in 293T cells using the second-generation packaging constructs pCMV-dR8.74 and pMD2G. Supernatants containing virus were collected at 48 hours and supplemented with 1 μg/ml polybrene before being used to infect cells for 6 hours. Knockdown efficiency was evaluated by Western blotting 48 hours post infection.

### Example 2

### Synergistic Combination of EGFR/HER2 Dual Tyrosine (TKI) Lapatinib and NF-κB Inhibitor CBL0137 in Stem and Non-Stem GBM Cells

FIG. 17 provides graphs showing that the combination of the EGFR/HER2 dual tyrosine inhibitor lapatinib and the NF-κB inhibitor CBL0137 provide synergistic results in stem and non-stem GBM cells. The first line graph shows that CBL0137 and lapatinib given in a 1:10 ratio provides increased inhibition in CD133+ GBM cells, while the second line graph shows that CBL0137 and lapatinib given in a 1:10 ratio provides increased inhibition in CD133− GBM cells. The bottom graph uses the Chou-Talalay combination index analysis described in Example 1 to confirm that the compounds were acting synergistically.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating cancer, comprising administering to a subject in need thereof a therapeutically effective amount of a tyrosine kinase inhibitor and an NF-κB inhibitor.

2. The method of claim 1, wherein the cancer includes an EGFR activating mutation.

3. The method of claim 1, wherein the subject has cancer that has developed resistance to tyrosine kinase inhibitors.

4. The method of claim 2, wherein the resistance has resulted from second site mutation of the EGFR receptor.

5. The method of claim 2, wherein the resistance has resulted from mutated KRAS in tumors with wildtype EGFR receptor or MET amplification.

6. The method of claim 1, wherein the cancer is non-small cell lung cancer.

7. The method of claim 1, wherein the tyrosine kinase inhibitor is erlotinib or gefitnib.

8. The method of claim 1, wherein the NF-κB inhibitor is a curaxin or a quinacrine derivative.

9. The method of claim 8, wherein the NF-κB inhibitor is quinacrine.

10. The method of claim 1, wherein the tyrosine kinase inhibitor and the NF-κB inhibitor are administered simultaneously.

11. The method of claim 1, wherein the subject is a human.

12. The method of claim 1, wherein the tyrosine kinase inhibitor and an NF-κB inhibitor are both administered in a pharmaceutically acceptable carrier.
